(19) United States
(12) Patent Application Publication
(10) Pub. No.: US 2015/0148617 A1
(43) Pub. Date: May 28, 2015

(54) METHOD AND SYSTEM FOR SELECTING ALARM REDUCTION ALGORITHM

(71) Applicant: General Electric Company, Schenectady, NY (US)

(72) Inventor: Bruce Arnold Friedman, Jasper, GA (US)

(73) Assignee: GENERAL ELECTRIC COMPANY, Schenectady, NY (US)

(21) Appl. No.: 14/091,653

(22) Filed: Nov. 27, 2013

(51) Int. Cl. A61B 5/00; G06F 19/00; A61B 5/0205

(52) U.S. Cl. CPC A61B 5/7275; A61B 5/02055; A61B 5/746; A61B 5/742; G06F 19/3406; G06F 19/327; A61B 5/021
USPC 600/301; 600/300; 600/485; 600/508; 600/529; 600/549; 600/484; 705/2

(57) ABSTRACT

A patient monitoring system and method is disclosed herein. The system includes one or more patient monitors that obtain physiological data from a patient. Based upon the physiological data obtained from the patient, as well as the information available for the patient in an electronic health record, an algorithm selection device determines which one of a plurality of early warning algorithms are best suited for use in monitoring a patient. One or more early warning algorithms can be presented to a user for selection. Once the algorithm selection device or the user determines which of a plurality of early warning algorithms would be most effective, the early warning algorithm is downloaded to the patient monitor for use by the patient monitor. The patient monitor utilizes the downloaded early warning algorithm to generate alarms and alerts to indicate the health status of the patient being monitored.

### BACKGROUND OF THE INVENTION

The present disclosure generally relates to a method and system for providing an early indication or warning of impending patient deterioration while reducing nuisance alarms. More specifically, the present disclosure relates to a method and system that is able to select one of a plurality of early warning algorithms to most accurately provide an early indication or warning, of an impending patient deterioration.

Presently available patient monitoring systems are able to monitor a relatively large number of different physiological data parameters obtained from a patient. in addition to monitoring the physiological data obtained from the patient, the patient monitors are able to retrieve stored information about the patient from an electronic health record. Further, the patient monitor allows the user to enter diagnostic information about the patient into the monitor. Thus, a clinician viewing the patient monitor is presented with what can be an overwhelming amount of changing data.

Because automatic monitoring of patients is becoming increasingly prevalent, various challenges have been identified. One of the principal challenges that result from automatic patient monitoring is alert fatigue. Alert fatigue is referred to as the condition in which clinicians become desensitized to clinical alerts because of the high probability that the alerts are not of actual clinical significance. Although one way to address this problem is to raise alert thresholds, this reduces sensitivity and increases the likelihood of failing to detect patients in deterioration.

As a result of the large amount of data available at the patient monitor, different types of early warning, algorithms have been developed to analyze the various different types of data and provide a single indication of the current health status of the patient. These various different algorithms operate utilizing different numbers and types of information to generate the assessment of the patient's overall health. The use of such algorithms allows for an early warning of the deteriorating health of the patient based upon the multiple parameters being monitored.

Although various different types of early warning algorithms have been developed to process various combinations of patient physiological data, patient demographic data and patient diagnostic data, each of these algorithms is specifically tailored for a certain type of available data as well as the type of patient being monitored. As an illustrative example, early warning algorithms have been developed for neonatal patients, which differ from algorithms developed for elderly or middle-aged patients. Therefore, in order for a patient monitor to accurately assess the overall health of a patient by utilizing an early warning algorithm, the patient monitor must be operating utilizing an algorithm that is appropriate for the patient being monitored.

### SUMMARY OF THE INVENTION

The present disclosure relates to a method and system for selecting and downloading an early warning algorithm for use with a patient monitor. The system and method identifies one of a plurality of an early warning algorithms that is most desirable for use with a patient monitor based upon at least one of patient demographic data, patient physiological data and patient diagnostic data that is available for the patient.

In accordance with the present disclosure, a patient monitor that is being used to monitor the status of a patient communicates with an algorithm selection device. The algorithm selection device accesses a computer database that includes the plurality of early warning algorithms. Based upon information available about the patient, the method computes a ranking of the early warning algorithms.

Once the plurality of early warning algorithms have been ranked, the method selects one of the early warning algorithms for use with the patient monitor. The selection of the early warning algorithm is based at least partially on one of the patient demographic data, the patient physiological data and the patient diagnostic data. The selection of the early warning algorithm can be done automatically by the algorithm selection device or can be done manually by a user.

After the most desirable early warning algorithm has been selected, the patient monitor operates using the selected early warning algorithm. In one embodiment, the selected early warning algorithm is downloaded to the patient monitor. Once the patient monitor receives the downloaded early warning algorithm, the patient monitor operates to monitor the patient and provide data to the early warning algorithm for analysis.

In another alternate embodiment, the selected early warning algorithm could also be executed in an algorithm server separate from the patient Monitor. In such a configuration, the patient monitor and the algorithm server would be configured to operate over the hospital network. Data obtained from the patient would be provided to the early warning algorithm over the hospital network for evaluation by the early warning algorithm residing on the algorithm server.

When the selection of the early warning algorithm is being made, the system and method utilizes various different parameters to determine which of the early warning algorithms may be most desirable for monitoring the patient. This selection can include determining What types of patient physiological data. In addition, the selection process can also be based on the types of patient demographic data and patient diagnostic data that may be available from an electronic health record for the patient. Once the early warning algorithms have been analyzed, the method displays the identified early warning algorithms to a user and allows the user to select one of the most preferred algorithms. The selection process can include ranking each of the early warning algorithms and displaying the ranking to a user such that the user can select the algorithm based upon the determined rankings.

The present disclosure also relates to a patient monitoring system for monitoring the status of a patient. The patient monitoring system includes at least one patient monitor that includes a display device and at least one sensor connected to the patient to obtain physiological data from the patient. The patient monitoring system further includes a computer database that includes a plurality of early warning algorithms. Each of the early warning algorithms operates to predict the deteriorating health of a patient based upon multiple parameters.

The patient monitoring system further includes an algorithm selection device that is in communication with the computer database and the patient monitor. The algorithm selection device includes a processor that is programmed to select one of the plurality of early warning algorithms based at least partially on the physiological data obtained from the patient. Further, the processor downloads the selected early warning algorithm to the patient monitor such that the patient monitor operates utilizing the downloaded early warning algorithm.

### BRIEF DESCRIPTION OF THE DRAWINGS

The drawings illustrate the best mode presently contemplated of carrying out the disclosure. In the drawings:

FIG. 1 is a schematic representation of a plurality of patient monitors that each operate using an early warning algorithm for patient monitoring in accordance with the present disclosure; and

FIG. 2 is a block diagram showing the method of identifying an early warning algorithm for use with a patient monitor.

### DETAILED DESCRIPTION OF THE INVENTION

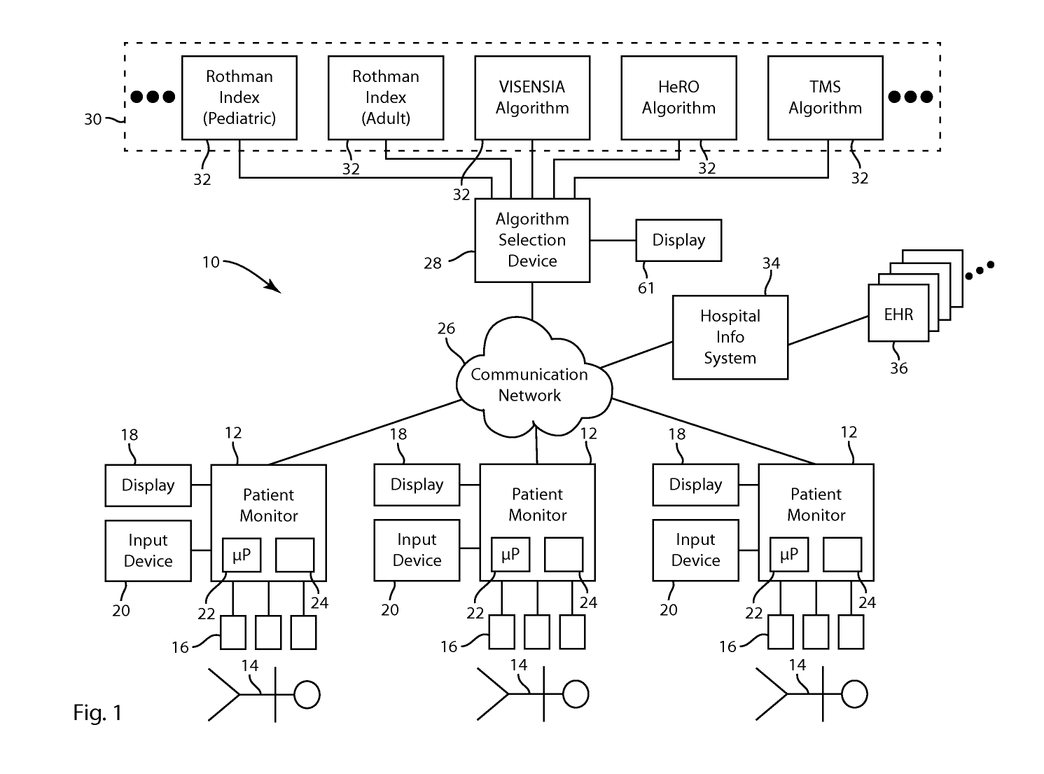

FIG. 1 illustrates a patient monitoring system 10 that operate in accordance with one embodiment of the present disclosure. The patient monitoring system 10 includes multiple patient monitors 12 that are each configured to monitor the health status of a patient 14. In the embodiment shown in FIG. 1, each patient monitor 12 includes a series of sensors 16 that obtain physiological data from the patient 14. As an illustrative example, the sensors 16 can obtain various different types of physiological data, including but not limited to blood pressure, temperature, respiration rate, SpO₂, values, ECG signal data, heart rate, as well as other physiological parameters that may be relevant to assessing the overall health of the patient 14. Since each of the patient monitors 12 are configurable devices, the sensors 16 can vary depending upon the clinical state of the patient 14 and the reasons for monitoring the health status of the patient.

Each of the patient monitors 12 further includes a display 18 that allows a controller 22 contained within the patient monitor 12 to present information to a user. Although the display 18 is shown separate from the patient monitor 12, it should be understood that the display 18 could be incorporated within the patient monitor while operating within the scope of the present disclosure.

In addition, each patient monitor 12 further includes an input device 20 that allows the user to enter various different data into the patient monitor 12 as well as to make selection choices based upon information shown on the display 18. The input device 20 can be various different types of components, such as a keyboard, mouse or any other device that allows information to be entered into the patient monitor 12. Further, it should be understood that the display 18 and input device 20 could be combined into a single device, such as a touch screen.

The patient monitor 12 includes a controller 22 and a memory device 24. The memory device 24 can store operating alarm limits, operating parameters as well as an algorithm for generating early warning alarms to a user. The controller 22 carries out the algorithm stored within the memory device 24 to generate alarms to a user, either through the display 18 or through other types of warning devices.

As illustrated in FIG. 1, each of the patient monitors 12 includes a bi-directional communication device that allows the patient monitor 12 to communicate over a communication network 26. The communication network 26 can be various different types of communication networks, such as the internet, a local area network, a wide area network, a wireless network, a virtual private network or the like. Through the communication network 26, each of the patient monitors is able to communicate with an algorithm selection device 28. The algorithm selection device 28 shown in FIG. 1 is a computer that is able to access a database 30 that includes a plurality of stored early warning algorithms 32. In the embodiment shown in FIG. 1, the computer database 30 includes five separate early warning algorithms 32. However, it should be understood that a significantly larger number of algorithms could be stored in the database 30 while operating within the scope of the present disclosure. In addition, a reduced number of early warning algorithms 32 could also be stored on the computer database 30.

The algorithm selection device 28 is also in communication with the hospital information system 34 through the communication network 26. The hospital information system 34 includes a database of patient information that includes a significant number of electronic health records (EHR) 36. Each EHR is stored for each of the patients 14 being monitored, as well as for all other patients that have been monitored in the hospital. The EHR 36 includes all of the information obtained from the patient over the lifetime of treatment of the patient at the facility. The EHR can include patient demographic data, historic patient physiological data and patient diagnostic data. As an illustrative example, the patient demographic data can include information relating to the patient's age, gender, family health history, race, height, weight and other types of parameters that are typically stored within an EHR 36. The patient diagnostic data can include information relating to past diagnostic assessments made for the patient, such as information relating to whether the patient is diabetic, obese, septic, or any other diagnostic assessments that have been made for the patient and may affect the current monitoring of the patient.

In addition to being accessible by the algorithm selection device 28, the hospital information system 34 can also be accessed by each of the patient monitors 12 through the communication network 26. Each of the patient monitors 12 may utilize the patient information contained in the EHR 36 to enhance the monitoring of the patient.

As can be seen and understood in FIG. 1, the algorithm selection device 28 is able to access the computer database 30 that includes all of the early warning algorithms 32. Each of the early warning algorithms 32 is designed and programmed to provide an early indication or warning of impending patient deterioration based upon a number of parameters obtained through a combination of the patient monitor 12 and the EHR 36. Table 1 set forth below indicates the different types of data and parameters used by each of the early warning algorithms 32.

TABLE 1

| | Patient Age | | | Data Type | | # | Parameters | | | | | |
|---|---|---|---|---|---|---|---|---|---|---|---|---|
| | Adult | Pediatric | Neonate | Episodic | Continuous | Parameters | SpO2 | Resp | ECG | NBP | Temp | IBP |
| Rothman Index | X | | | X | | 26 | | | | | | |
| (adult) | | | | | | | | | | | | |
| Rothman index | | X | | X | | 26 | | | | | | |
| (pediatric) | | | | | | | | | | | | |
| VISENSIA | X | X | | X | | 5 | X | X | X | X | X | |
| HeRO | | | X | | X | 1 | | | X | | | |
| TMS | X | | | | X | 3 | | X | X | | | X |

As Shown in Table 1, the Rothman index (adult) is an early warning algorithm that is particularly useful with adult patients based upon episodic data. The Rothman index utilizes twenty-six different parameters in generating an overall index. The twenty-six patient indicators are all obtained from the EHR 36 for the patient. These parameters obtained from the EHR 36 can include historic vital sign information, pulse oximeter data, lab values, a Braden score, individual nursing assessments, as well as other data included in the EHR 36. Based upon the twenty-six parameters obtained, the Rothman early warning algorithm generates an index score that provides an early warning indication of the patient's health.

In addition to the Rothman index for adult patients, the database 30 further includes an early warning algorithm that generates a Rothman index for pediatric patients. The Rothman index for pediatric patients also uses twenty-six different parameters. However, since pediatric patients have different parameters that may indicate deteriorating health, it is desirable to use the early warning algorithm that generates the Rothman index for pediatric patients when the patient monitor is being used to monitor a pediatric patient.

The next early warning algorithm 32 shown in FIG. 1 and Table 1 above is the VISENSIA algorithm that is available from Oxford Biosystems. The VISENSIA. algorithm can be used with both adult and pediatric patients and utilizes episodic data. As illustrated in Table 1, the VISENSIA algorithm utilizes five separate physiological parameters from the patient, including SpO₂, respiration rate, ECG signal data, noninvasive blood pressure measurements and patient temperature. The VISENSIA algorithm thus does not utilize information from the EHR 36. The VISENSIA algorithm may be most desirable when the patient monitor includes sensors that monitor most or all of the five physiological parameters identified above and the information in the EHR is either incomplete or sparsely populated.

The next algorithm shown in FIG. 1 and Table 1 is the HeRO algorithm. The HeRO algorithm is particularly desirable for use with neonatal patients and utilizes continuous data for operating the patient monitor 12. The HeRO algorithm utilizes only a single parameter, namely ECG signals obtained continuously from the patient.

The final algorithm in FIG. 1 and Table 1 is the TMS algorithm. The TMS algorithm is particularly useful with adult patients and utilizes continuous data. The continuous data from the patient includes respiration rate, invasive blood pressure, and ECG signal data.

As can be understood by Table 1 and the above description, the algorithm selection device 28 shown in FIG. 1 can select one or more of the early warning algorithms 32 based upon the physiological data available from the patient monitor 12 connected to the patient as well as either the patient biographic data or patient diagnostic data that is available in the EHR 36. Based upon the availability of this information, the algorithm selection device 28 selects the most appropriate algorithm and makes the algorithm accessible for use in operating the patient monitor 12.

Set forth below in Table 2 is a sample weighting system that can be utilized by the algorithm selection device 28 in selecting which of the early warning algorithms 32 is most beneficial for monitoring an individual patient. In Table 2, a weight is assigned for each type of the patient demographic data and patient physiological data that is available from either the patient monitor 12 or the ERR 36. As can be seen below, the type of data, whether continuous or episodic, is a factor used to select between the early warning algorithms. Based upon a combined score of the parameters that are available for use, the algorithm selection device can use a compiled score for each algorithm to rank and ultimately select the one or more early warning algorithms that are most applicable for the patient.

TABLE 2

| | Patient Age | | | Data Type | | # | Parameters | | | | | |
|---|---|---|---|---|---|---|---|---|---|---|---|---|
| | Adult | Pediatric | Neonate | Episodic | Continuous | Parameters | SpO2 | Resp | ECG | NBP | Temp | IBP |
| Rothman Index | 5 | 0 | 0 | 5 | 0 | 26 | 0 | 0 | 0 | 0 | 0 | 0 |
| (adult) | | | | | | | | | | | | |
| Rothman index | 0 | 5 | 0 | 5 | 0 | 26 | 0 | 0 | 0 | 0 | 0 | 0 |
| (pediatric) | | | | | | | | | | | | |
| VISENSIA | 5 | 3 | 1 | 5 | 4 | 5 | 3 | 4 | 2 | 3 | 1 | 0 |
| HeRO | 0 | 2 | 5 | 0 | 5 | 1 | 0 | 0 | 5 | 0 | 0 | 0 |
| TMS | 5 | 3 | 1 | 0 | 5 | 3 | 0 | 3 | 3 | 0 | 0 | 3 |

Once the most desirable algorithm is selected, the algorithm utilized to control the operation of the patient monitor 12. In one embodiment, the selected early warning algorithm is downloaded to the memory device 24 contained within the patient monitor 12. Thus, the patient monitor 12 can be delivered without any pre-stored algorithm. Further, the patient monitor 12 can obtain additional or alternate early warning algorithms 32 when the patient monitor is used to monitor different types of patients within a hospital environment. In this manner, the patient monitor 12 can be configured during use and most effectively provide an early warning algorithm 32 based upon the patient being monitored.

In an alternate embodiment, the selected early warning algorithm can be operated on an algorithm server that is separate from the patient monitor. The algorithm server could be part of the algorithm selection device of could be a separate component. In this embodiment, the patient monitor communicates the monitored patient physiological data to the algorithm server over the hospital network. The early warning algorithm would operate using the physiological data and the other available patient diagnostic or demographic data and communicate back to the patient monitor through the hospital network.

Although the selection of the algorithm is made in the algorithm selection device 28, it is contemplated that such determination could also be made at the patient monitor 12 using the controller 22. However, the embodiment and configuration shown in FIG. 1 is thought to be more preferred since the algorithm selection device 28 can be located separate from the multiple patient monitors 12 and thus be used across each of the multiple patient monitors.

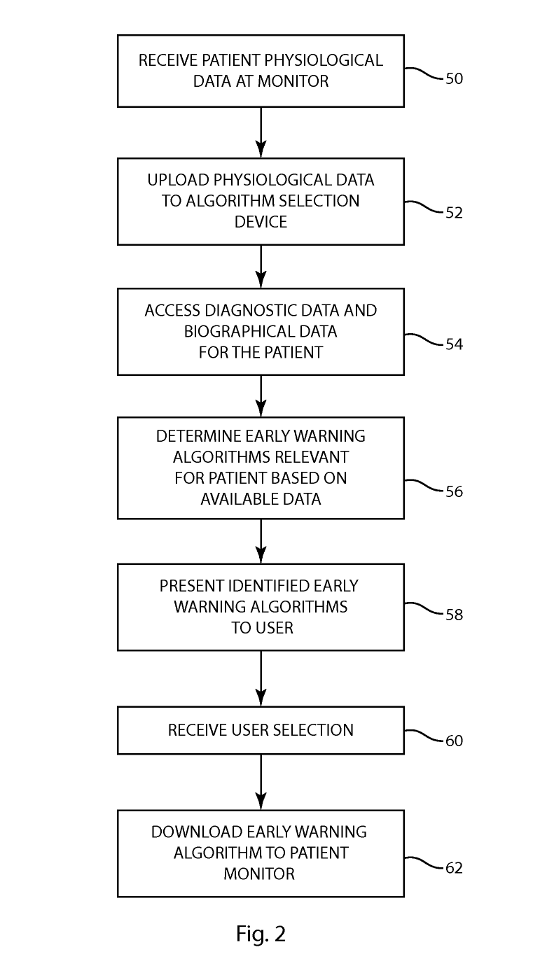

FIG. 2 illustrates the operational steps and sequence utilized to early out the function and control of the patient monitoring system 10 shown in FIG. 1. In step 50, the patient monitor receives patient physiological data directly from the sensors 16 connected to the patient. As indicated above, the physiological data obtained at the patient monitor can be widely varied depending upon the type of sensors utilized. Examples of physiological data obtained from the patient can include blood pressure, heart rate, respiration rate, temperature and ECG signal data.

Once this data is obtained at the patient monitor, the existence of the physiological data is reported up to the algorithm selection device through the communication network, as indicated in step 52. Once the algorithm selection device receives information about the type of physiological data that is available through the sensors, the algorithm selection device communicates to the hospital information system and determines what type of diagnostic data and demographic data are available for the patient from the electronic health record, as indicated in step 54. As previously described, various different types of diagnostic data and demographic data can be available for the patient in the EHR. However, if the patient has not been in a clinical or hospital environment, the EHR may not include any information and thus various different early warning algorithms may not be applicable for use with that patient.

Once the algorithm selection device has obtained information about the types of physiological data, demographic data and diagnostic data that is available for the patient being monitored at the patient monitor, the algorithm selection device determines in step 56 which of the plurality of early warning algorithms may be relevant for the patient. If the EHR is complete and multiple sensors are being used with the patient, more than one of the early warning algorithms may be particularly useful in monitoring a patient. Alternatively, if the EHR is not complete and only very little physiological data is being obtained from the patient, only one of the multiple early warning algorithms may be desirable for use with the patient.

In step 58, the algorithm selection device 28 displays a list of early warning algorithms that could be used with the patient monitor. This list of possible early warning algorithms can include a ranking indicating which of the early warning algorithms appears to be most desirable for use with the patient monitor. This ranking can be determined based upon the scoring system shown in Table 2 or can be based upon other parameters. The list of possible early warning algorithms and a ranking of the algorithms can be presented to the user either on a display 61 associated with the algorithm selection device or on the display 18 associated with the patient monitor. Since the user most likely will be at the patient monitor 12, it is contemplated that the listing of possible early warning algorithms will be presented on the display 18 at the patient monitor.

Alternatively, instead of presenting information to the user, the algorithm selection device 28 could automatically select the most preferred early warning algorithm 32 without requiring any user input. Although the automatic selection of the early warning algorithm may remove any incorrect selection by the user, it is contemplated that presenting the selection information to the user would allow the user to make informed decisions about the type of algorithm being utilized to operate the patient monitor.

In step 60, the algorithm selection device receives a selection by the user of the desired early warning algorithm for use in the patient monitor. The selection made by the user can be through a user input device 20 associated with the patient monitor or through a similar input device associated with the algorithm selection device 28 shown in FIG. 1.

Once the user selects the early warning algorithm 32, the early warning algorithm is downloaded to the patient monitor, as indicated in step 62. The early warning algorithm is downloaded through the communication network 26 and is stored within a memory device 24 associated with the patient monitor. Once the early warning algorithm has been downloaded, the patient monitor 12 operates utilizing the early warning algorithm and generates patient alerts/warnings based upon the operation of the early warning algorithm.

If the patient monitor is used with a different type of patient or with a different grouping of sensors, the patient monitor can again communicate back to the algorithm selection device for the algorithm selection device to select the early warning algorithm that is most relevant, as was described with reference to step 56 shown in FIG. 2. In this manner, the patient monitor is able to download only the early warning algorithm that is most desirable for the patient being monitored and allows the patient monitor to adjust the algorithm if the monitor is used with a different type of patient.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for automatically ranking the effectiveness of early warning algorithms for a patient, comprising:
accessing a computer database comprising a plurality of early warning algorithms;
computing a ranking of at least a subset of the plurality of early warning algorithms based on the type of available patient data; and
providing the ranking for a predetermined use.

2. The method of claim 1, wherein the patient data comprises of at least one of patient demographic data, patient physiological data and patient diagnostic data.

3. The method of claim 2, wherein the patient demographic data comprises at least one of patient age, patient gender, family history and race.

4. The method of claim 2, wherein patient physiological data comprises at least one of blood pressure, heart rate, respiration rate, and temperature.

5. The method of claim 1, wherein the type of patient data further comprises whether the data is continuous or episodic.

6. The method of claim 1 wherein the patient diagnostic information comprises at least one of current or prior pathological conditions.

7. The method of claim 1, further comprising the step of displaying the ranking to a user on a display device.

8. A method of operating, a patient monitor to generate an early warning of deteriorating patient status, comprising:
acquiring physiological data from the patient at the patient monitor;
accessing a computer database external to the patient monitor and including a plurality of early warning algorithms;
selecting and downloading one of the plurality of early warning algorithms based at least partially on the physiological data acquired by the patient monitor; and
operating the patient monitor utilizing the downloaded early warning algorithm.

9. The method of claim 8 further comprising the step of acquiring patient demographic data for the patient, wherein the step of selecting one of the early warning, algorithms is based on both the acquired patient demographic data and the acquired physiological data.

10. The method of claim 9 wherein the patient demographic data comprises at least one of patient age, patient gender, family history and race.

11. The method of claim 8 wherein the physiological data from the patient includes at least one of blood pressure, heart rate, respiration rate and temperature.

12. The method of claim 8 wherein the step of selecting the early warning algorithm comprises:
identifying one or more of the plurality of early warning algorithms best suited for use in monitoring the patient;
displaying the identified early warning algorithms to a user; and
receiving a selection of one of the displayed early warning algorithms.

13. The method of claim 12 further comprising the steps of:
generating a ranking for each of the identified early warning algorithms; and
displaying the ranking to the user.

14. The method of claim 13 wherein the rankings are based upon the availability of the physiological data and patient demographic data.

15. The method of claim 9 further comprising the step of acquiring patient diagnostic data. wherein the step of selecting one of the early warning algorithms is further based on the acquired patient diagnostic data.

16. A patient monitoring system for monitoring the status of a patient, comprising:
a patient monitor including a display device and at least one sensor connected to the patient to obtain physiological data from the patient;
a computer database external to the patient monitor and including a plurality of early warning algorithms; and
an algorithm selection device in communication with the computer database, wherein the algorithm selection controller includes a processor programmed to select and download one of the plurality of early warning algorithms based at least partially on the physiological data obtained from the patient,
wherein the patient monitor operates utilizing the downloaded early warning algorithm.

17. The patient monitoring system of claim 16 wherein the processor is further programmed to identify one or more of the plurality of early warning algorithms best suited for use in monitoring the patient and displaying the identified early warning algorithms to a user.

18. The patient monitoring system of claim 16 wherein the physiological data includes at least one of blood pressure, heart rate, respiration rate and temperature.

19. The patient monitoring system of claim 16 wherein the processor selects the one or more early warning algorithm based upon the physiological data, patient demographic data and patient diagnostic data.

20. The patient monitoring system of claim 16 wherein the processor is further programmed to display the identified early warning algorithms to a user and receive a selection of one of the displayed early warning algorithms from the user.
